(19) United States
(12) Patent Application Publication
(10) Pub. No.: US 2019/0029496 A1
(43) Pub. Date: Jan. 31, 2019

(54) METHOD FOR MANUFACTURING OPTICAL UNIT FOR ENDOSCOPE AND ENDOSCOPE

(71) Applicant: OLYMPUS CORPORATION, Tokyo (JP)

(72) Inventor: Takatoshi IGARASHI, Ina-shi (JP)

(73) Assignee: OLYMPUS CORPORATION, Tokyo (JP)

(21) Appl. No.: 16/151,480

(22) Filed: Oct. 4, 2018

Related U.S. Application Data
(63) Continuation of application No. PCT/JP2016/061870, filed on Apr. 13, 2016.

(51) Int. Cl. A61B 1/00; G02B 7/02; A61B 1/04; H01L 27/14; H04N 5/225

(52) U.S. Cl. CPC A61B 1/0011; G02B 7/02; H04N 5/335; H01L 27/14; H04N 5/225; A61B 1/04

(57) ABSTRACT

A method for manufacturing optical units for endoscope includes: a step of fabricating device wafers; a step of laminating the device wafers to fabricate a bonded wafer; a first fixation step of fixing the bonded wafer to a first substrate; a first cutting step of cutting the bonded wafer along first cutting lines to divide the bonded wafer into slice bodies; a second fixation step of fixing cutting surfaces of the slice bodies to a second substrate; a second cutting step of cutting the slice bodies along second cutting lines to divide the slice bodies into the optical units for endoscope; and a step of removing the optical units for endoscope from the second substrate; and an area of each side face fixed to the second substrate is larger than an area of a light incident surface.

### CROSS REFERENCE TO RELATED APPLICATION

This application is a continuation application of PCT/JP2016/061870 filed on Apr. 13, 2016, the entire contents of which are incorporated herein by this reference.

### BACKGROUND OF THE INVENTION

### Field of the Invention

The present invention relates to a method for manufacturing an optical unit for endoscope in which a plurality of devices are laminated, and an endoscope including the optical unit for endoscope in a rigid distal end portion.

### Description of the Related Art

It is important for an optical unit for endoscope arranged in a rigid distal end portion of an endoscope to be downsized for minimization of invasiveness. For example, an area of a light incident surface is several mm² or smaller, and an area of a very small light incident surface is 1 mm² or smaller. As a method for manufacturing an extremely small optical unit, there is a method in which a bonded wafer is fabricated by laminating a plurality of device wafers each of which includes a plurality of optical elements, and the bonded wafer is cut and divided. The bonded wafer is cut, for example, after being adhesively fixed to a dicing tape.

Note that the optical unit manufacturing method described above is similar to a multi-memory module manufacturing method disclosed in Japanese Patent Application Laid-Open Publication No. 2014-71932.

In the case of the optical unit with a light incident surface area of several mm² or smaller, especially in the case of the optical unit with a light incident surface area of 1 mm² or smaller, an area of being adhesively fixed to a dicing tape is also a very small area of several mm² or smaller, especially, 1 mm² or smaller. Therefore, it is not easy to sufficiently fix the optical unit.

### SUMMARY OF THE INVENTION

A method for manufacturing optical units for endoscope of an embodiment of the present invention includes: a step of fabricating a plurality of device wafers including at least one optical element wafer, each of the plurality of device wafers including a plurality of devices; a step of laminating the plurality of device wafers to fabricate a bonded wafer; a first fixation step of fixing a main face of the bonded wafer to a first substrate; a first cutting step of cutting the bonded wafer along mutually parallel first cutting lines to divide the bonded wafer into slice bodies; a step of removing the slice bodies from the first substrate; a second fixation step of fixing cutting surfaces of the slice bodies to a second substrate; a second cutting step of cutting the slice bodies along mutually parallel second cutting lines orthogonal to the first cutting lines to divide the slice bodies into the optical units for endoscope; and a step of removing the optical units for endoscope from the second substrate; and an area of a side face of each of the optical units for endoscope is larger than an area of a light incident surface, the side face being fixed to the second substrate and being orthogonal to the light incident surface.

An endoscope of an embodiment of the present invention includes an optical unit for endoscope in a rigid distal end portion of an insertion portion; the optical unit for endoscope is manufactured by a manufacturing method including: a step of fabricating a plurality of device wafers including at least one optical element wafer, each of the plurality of device wafers including a plurality of devices; a step of laminating the plurality of device wafers to fabricate a bonded wafer; a first fixation step of fixing a main face of the bonded wafer to a first substrate; a first cutting step of cutting the bonded wafer along mutually parallel first cutting lines to divide the bonded wafer into slice bodies; a step of removing the slice bodies from the first substrate; a second fixation step of fixing cutting surfaces of the slice bodies to a second substrate; a second cutting step of cutting the slice bodies along mutually parallel second cutting lines orthogonal to the first cutting lines to divide the slice bodies into the optical units for endoscope; and a step of removing the optical units for endoscope from the second substrate; and an area of a side face of each of the optical units for endoscope is larger than an area of a light incident surface, the side face being fixed to the second substrate and being orthogonal to the light incident surface.

### BRIEF DESCRIPTION OF THE DRAWINGS

FIG. 1 is a perspective view of an image pickup unit of a first embodiment;

FIG. 2 is a cross-sectional view of the image pickup unit of the first embodiment along a II-II line in FIG. 1;

FIG. 3 is a perspective view of an endoscope of the first embodiment;

FIG. 4 is a flowchart for illustrating a method for manufacturing the image pickup units of the first embodiment;

FIG. 5 is an exploded view for illustrating the method for manufacturing the image pickup units of the first embodiment;

FIG. 6 is a perspective view for illustrating the method for manufacturing the image pickup units of the first embodiment;

FIG. 7 is a perspective view for illustrating the method for manufacturing the image pickup units of the first embodiment;

FIG. 8 is a perspective view for illustrating the method for manufacturing the image pickup units of the first embodiment;

FIG. 9 is a perspective view for illustrating the method for manufacturing the image pickup units of the first embodiment;

FIG. 10 is a perspective partial cross-sectional view of a device wafer for illustrating the method for manufacturing the image pickup units of the first embodiment;

FIG. 11 is a perspective view of a slice body for illustrating the method for manufacturing the image pickup units of the first embodiment;

FIG. 12A is a cross-sectional view for illustrating a method for manufacturing image pickup units of a first modification of the first embodiment;

FIG. 12B is a cross-sectional view for illustrating the method for manufacturing the image pickup units of the first modification of the first embodiment;

FIG. 13A is a cross-sectional view for illustrating a method for manufacturing image pickup units of a second modification of the first embodiment;

FIG. 13B is a cross-sectional view for illustrating the method for manufacturing the image pickup units of the second modification of the first embodiment;

FIG. 14A is a cross-sectional view for illustrating a method for manufacturing image pickup units of a third modification of the first embodiment;

FIG. 14B is a cross-sectional view for illustrating the method for manufacturing the image pickup units of the third modification of the first embodiment;

FIG. 14C is a perspective view of an image pickup unit of the third modification of the first embodiment;

FIG. 15A is a cross-sectional view for illustrating a method for manufacturing lens units of a second embodiment;

FIG. 15B is an exploded view of an image pickup apparatus that includes the lens unit of the second embodiment;

FIG. 16A is a cross-sectional view for illustrating a method for manufacturing lens units of a first modification of the second embodiment;

FIG. 16B is an exploded view of an image pickup apparatus that includes the lens unit of the first modification of the second embodiment;

FIG. 17A is a cross-sectional view for illustrating a method for manufacturing lens units of a second modification of the second embodiment;

FIG. 17B is a cross-sectional view for illustrating the method for manufacturing the lens units of the second modification of the second embodiment;

FIG. 17C is a cross-sectional view for illustrating the method for manufacturing the lens units of the second modification of the second embodiment;

FIG. 17D is a cross-sectional view for illustrating the method for manufacturing the lens units of the second modification of the second embodiment; and

FIG. 17E is a cross-sectional view for illustrating the method for manufacturing the lens units of the second modification of the second embodiment.

### DETAILED DESCRIPTION OF THE PREFERRED EMBODIMENTS

### First Embodiment

### <Configuration of Image Pickup Unit>

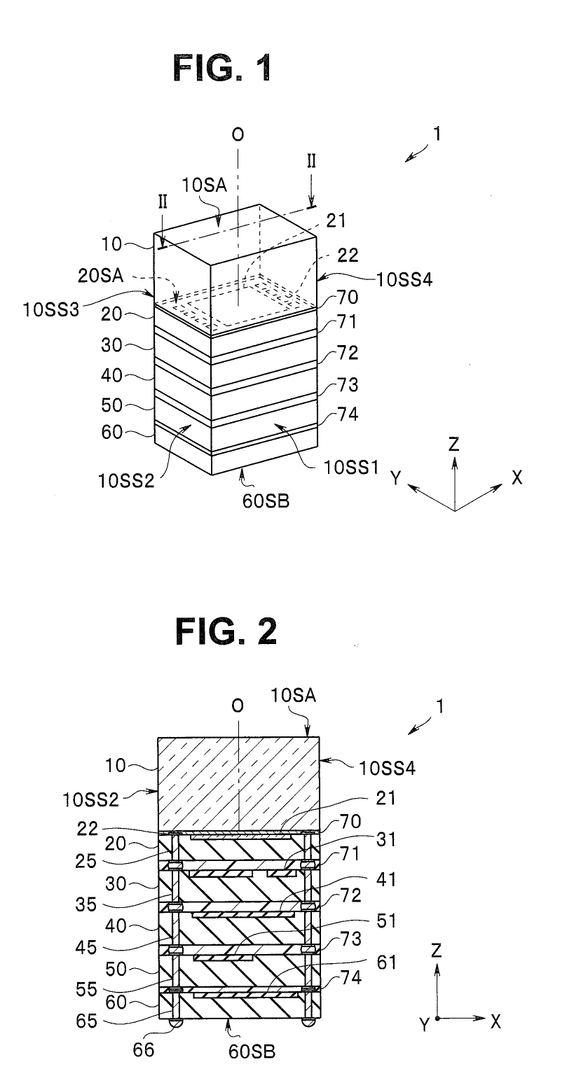

As shown in FIGS. 1 and 2, an optical unit for endoscope of the present embodiment is an image pickup unit (an image pickup apparatus) 1 in which an image pickup device 20 and a plurality of semiconductor devices 30 to 60 are laminated.

Note that all drawings are schematic, and it should be noticed that a relationship between thickness and width of each portion, a thickness ratio among respective portions and the like are different from actual ones; and, among the drawings, portions having a different mutual dimensional relationship or ratio may be included. Further, there may be a case where some components are not shown.

The image pickup unit 1 is configured with a cover glass element 10, an image pickup device (an imager) 20 and semiconductor devices 30, 40, 50 and 60 having same-size cross sections in a direction orthogonal to an optical path (an optical axis O) and being laminated in that order. As described later, the image pickup unit 1 is a wafer-level optical unit fabricated by cutting a bonded wafer in which a plurality of wafers are laminated, and an external form of the image pickup unit 1 is a rectangular parallelepiped.

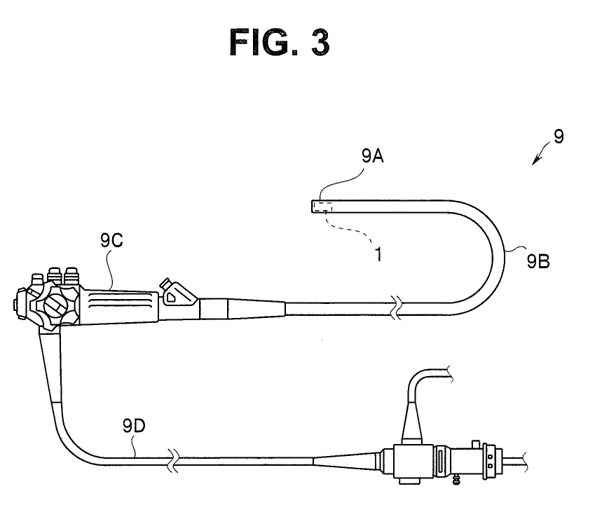

As shown in FIG. 3, the image pickup unit 1 is arranged in a rigid distal end portion 9A of an insertion portion 9B of an endoscope 9 to pick up an object image, and process and output an image pickup signal. The endoscope 9 of another embodiment includes the insertion portion 9B in which the image pickup unit 1 is arranged in the rigid distal end portion 9A, an operation portion 9C arranged on a proximal end side of the insertion portion 9B, and a universal cord 9D extending from the operation portion 9C. Note that an image pickup signal outputted from the image pickup unit 1 arranged in the rigid distal end portion 9A is transmitted to a processor via a cable inserted through the universal cord 9D. A drive signal to the image pickup unit 1 is also transmitted from the processor via a cable inserted through the universal cord 9D.

The cover glass element 10 is formed with transparent material that protects an image pickup surface of the image pickup device 20. The image pickup device 20 and the semiconductor devices 30 to 60 are configured with semiconductors such as silicon.

On an image pickup surface 20SA of the image pickup device 20, a light receiving portion 21 such as a CMOS light receiving element, and electrodes 22 connected to the light receiving portion 21 are formed. The electrodes 22 are connected to electrodes on a back face opposite to the image pickup surface 20SA via a through wiring 25. On the image pickup surface 20SA, the cover glass element 10 is attached via transparent adhesive resin 70.

Semiconductor circuits 31 to 61 are formed on the semiconductor devices 30 to 60, respectively. The semiconductor devices 30 to 60 are mutually connected via through wirings 35, 45, 55 and 65. On a back face 60SB of the semiconductor device 60, a bump 66 connected to the through wiring 65 is arranged. The image pickup unit 1 receives and transmits electrical signals via the bump 66.

Among the image pickup device 20 and the semiconductor devices 30 to 60, insulating resin 71 to 74 is filled for mechanical reinforcement and improvement of bonding reliability.

The image pickup unit 1 is in a rectangular parallelepiped shape having a light incident surface 10SA, the back face 60SB and four side faces 10SS1 to 10SS4.

A cross section orthogonal to the optical axis O, for example, the light incident surface 10SA is in a rectangular shape of 0.7 mm×0.5 mm. That is, an area S1 of the light incident surface 10SA is only 0.35 mm². Note that a height (a dimension in a Z direction) of the image pickup unit 1 is 1.5 mm. Therefore, areas (S2 and S3) of side faces 10SS1 to 10SS4 are S2=1.05 mm² and S3=0.75 mm², which are larger than the area S1 (0.35 mm²) of the light incident surface 10SA.

Though the area S1 of the light incident surface 10SA of the image pickup unit 1 is 0.35 mm² that is smaller than 1 mm², there is not a possibility that image pickup units 1 which have been cut come off from a dicing tape and are scattered or that it is not possible to cut along a desired cutting line, during cutting, because the image pickup units 1 are manufactured by a manufacturing method to be described later. Therefore, productivity of the image pickup units 1 is high. Note that the present invention is especially effective for such an image pickup unit that the area S1 of the light incident surface 10SA is 1 mm² or smaller.

### <Image Pickup Unit Manufacturing Method>

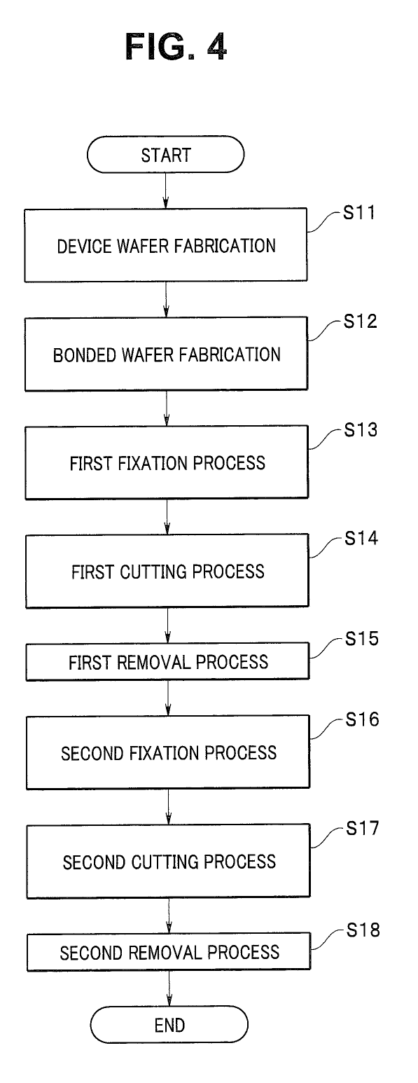

Next, a method for manufacturing image pickup units of the embodiment will be described along a flowchart shown in FIG. 4.

### <Step S11> Device Wafer Fabrication

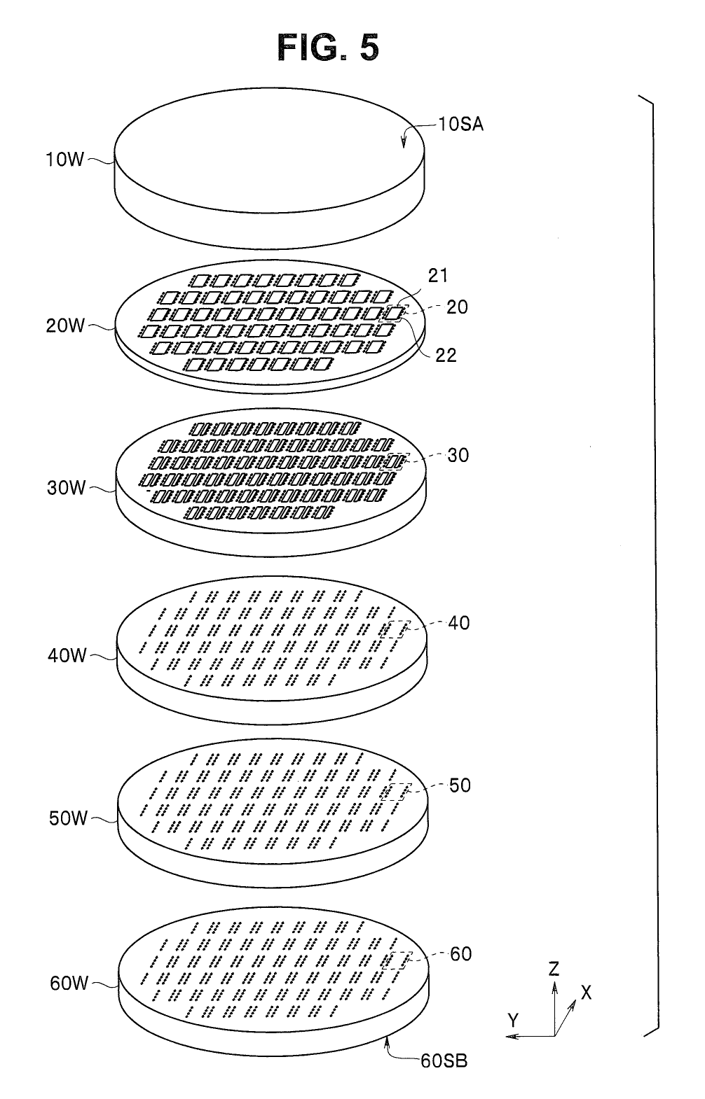

As shown in FIG. 5, a plurality of device wafers 10W to 60W including at least one optical element wafer are fabricated, each of the plurality of device wafers including a plurality of devices.

The device wafer 10W is a glass wafer and can be regarded as an optical element wafer that includes a plurality of cover glass elements 10. The device wafer 10W is only required to be transparent in a light wavelength band for image pickup, and, for example, borosilicate glass, quartz glass, or single crystal sapphire is used.

The image pickup wafer 20W includes a plurality of image pickup devices 20, the light receiving portion 21 and the like being formed on each of the plurality of image pickup devices 20 by a well-known semiconductor manufacturing technique. Readout circuits may be formed on the image pickup wafer 20W. On each of the semiconductor wafers 30W to 60W, a plurality of semiconductor circuits are formed by a well-known semiconductor manufacturing technique. On the image pickup devices 20 of the image pickup wafer 20W and the semiconductor devices 30 to 60 of the semiconductor wafers 30W to 60W, respectively, through wirings 25 to 65 are formed, respectively. The through wirings 25 to 65 may be formed after the plurality of device wafers 10W to 60W are laminated in a bonded wafer fabrication process to be described later.

For example, each semiconductor circuit 31 of the semiconductor wafer 30W includes a plurality of thin film capacitors and performs primary processing of an image pickup signal outputted by the light receiving portion 21. Each semiconductor circuit 41 of the semiconductor wafer 40W performs AD conversion processing of the image pickup signal outputted by the semiconductor circuit 31. Each semiconductor circuit 51 of the semiconductor wafer 50W includes a transmission buffer, a resistance and a capacitor. Each semiconductor circuit 61 of the semiconductor wafer 60W includes a timing adjusting circuit. The number of semiconductor wafers, the kind of semiconductor circuit included in each of the semiconductor wafers, and the like are set according to specifications of the image pickup unit 1. The semiconductor circuits may be formed on both faces of each semiconductor wafer, or the semiconductor circuits may be formed on a back face of each semiconductor wafer.

### <Step S12> Bonded Wafer Fabrication

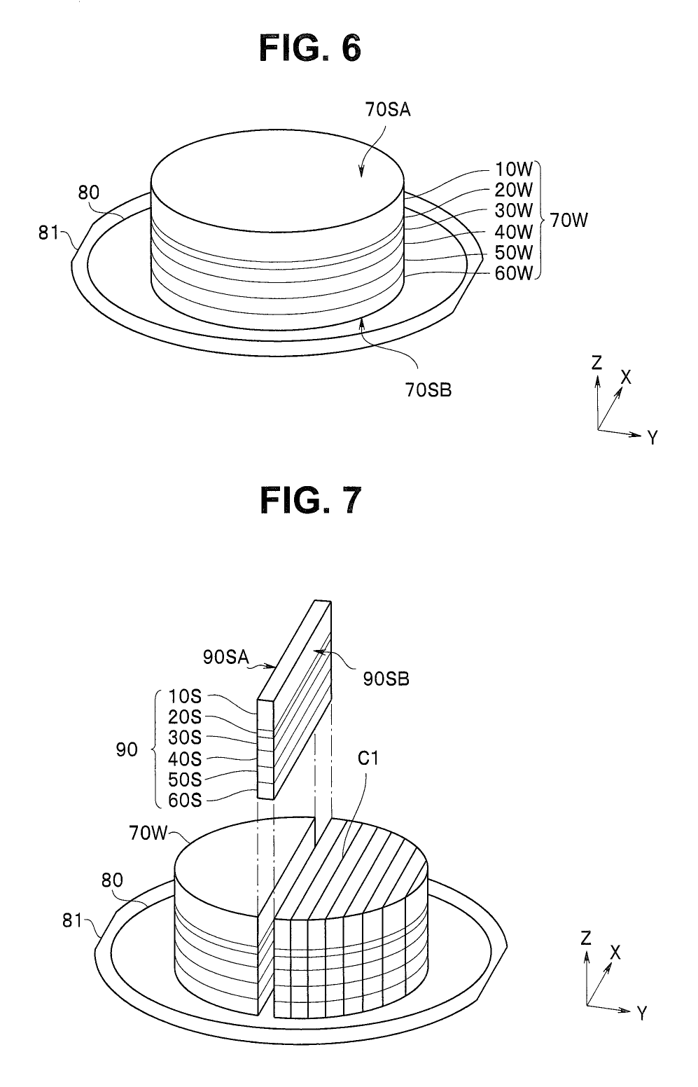

As shown in FIG. 6, the plurality of device wafers 10W to 60W are laminated to fabricate a bonded wafer 70W. When the device wafers 10W to 60W are laminated, the devices of the image pickup wafer 20W and the semiconductor wafers 30W to 60W are electrically connected via the through wirings 25 to 65, respectively. The transparent adhesive resin 70 is filled between the device wafer 10W, which is a cover glass wafer, and the image pickup wafer 20W, and the insulating resin 71 to 74 is filled among the image pickup device 20 and the semiconductor devices 30 to 60, though it is not shown in the drawings below.

Electrical connection among the wafers may be provided by bump electrodes. Otherwise, the wafers may be electrically connected by through wirings after mechanically bonding the respective wafers by direct bonding via insulating films. The respective wafers may be connected by hybrid bonding in which the insulating films and the connection electrodes are collectively connected, via the connection electrodes embedded in the insulating films.

### <Step S13> First Fixation Process

A main face 70SB of the bonded wafer 70W is adhesively fixed to a dicing tape 80 which is a first substrate. Note that the dicing tape 80 is held by a dicing frame 81. The first substrate is not limited to the dicing tape 80 if the bonded wafer 70W can be fixed. The main face 70SA of the bonded wafer 70W may be fixed to the dicing tape 80. Furthermore, instead of adhesive fixation, the bonded wafer 70W may be fixed using wax.

### <Step S14> First Cutting Process

As shown in FIG. 7, the bonded wafer 70W is cut along a plurality of mutually parallel first cutting lines C1, for example, by a dicing saw and divided into slice bodies 90. Side faces of each of the slice bodies 90 are formed by cutting surfaces 90SA and 90SB. Laser dicing or plasma dicing may be used for cutting.

### <Step S15>

The plurality of slice bodies 90 are removed from the dicing tape 80 which is the first substrate. Since the adhesive force of the dicing tape 80 disappears, for example, when the dicing tape 80 is irradiated by ultraviolet rays or heated, the slice bodies 90 can be easily separated from the dicing tape 80.

### <Step S16> Second Fixation Process

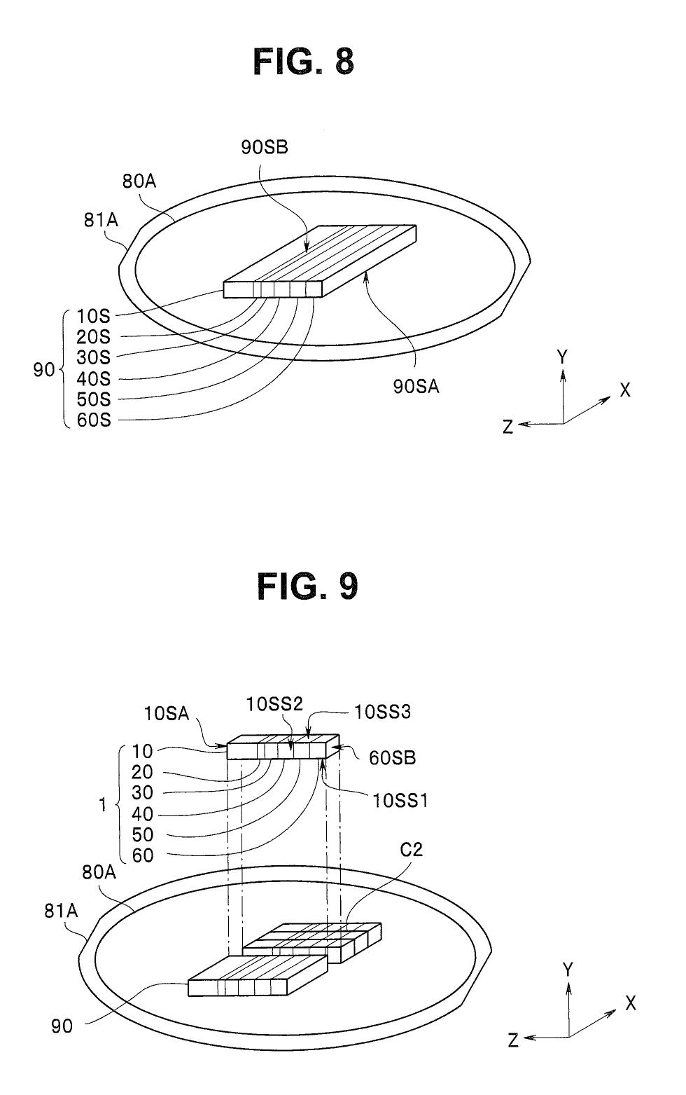

As shown in FIG. 8, the cutting surface 90SA of each slice body 90 is adhesively fixed to a dicing tape 80A which is a second substrate. Note that the dicing tape 80A is held by a dicing frame 81A. The cutting surface 90SB of the slice body 90 may be fixed to the dicing tape 80A.

Note that the dicing tape 80 and the dicing tape 80A may be dicing tapes of a same kind or may be different kinds of fixing members.

### <Step S17> Second Cutting Process

As shown in FIG. 9, the slice body 90 is cut along a plurality of mutually parallel second cutting lines C2 orthogonal to the first cutting lines C1 and divided into image pickup units 1 which are optical units for endoscope. A method for the second cutting may be the same as or different from the method for the first cutting. For example, the method for the first cutting and the method for the second cutting may be a dicing saw and laser dicing, respectively.

As already described, the area S1 of the light incident surface 10SA of each image pickup unit 1, which is a rectangular parallelepiped, is 0.35 mm² that is smaller than 1 mm². However, in the second cutting process, each of the cut image pickup units 1 is fixed to the dicing tape 80A by the side face 10SS1 the area of which is larger than an area of the light incident surface 10SA. That is, the area S2 of the side face 10SS1 is 1.05 mm², which is three times as large as the area S1.

Since the fixation area is large, during the cutting process the image pickup units 1 which have been cut do not come off from the dicing tape 80A nor are scattered, or it is not possible for the image pickup units 1 not to be cut along a desired cutting line. Therefore, productivity of the image pickup units 1 is high.

Note that in the case of such image pickup units 1 that the area S2 of the side face 10SS orthogonal to the light incident surface 10SA is larger than the area S1 of the light incident surface 10SA, the manufacturing method of the present embodiment has the effects described above. From a viewpoint of productivity, it is preferable that a lower limit of the area S1 of the light incident surface 10SA is, for example, 0.05 mm² or larger. Note that it is preferable that the area S2 of the side face 1055 is 1.5 times or more larger than the area S1 of the light incident surface 10SA, and it is especially preferable that the area S2 is 2.0 times or more larger. Further, it is preferable that the area S2 of the side face 10SS is more than 1 mm²

Furthermore, the larger the area of the face of each cut image pickup unit 1 fixed to the dicing tape 80A is, the higher the productivity is. Therefore, in a case where the light incident surface 10SA of each of the image pickup units 1 divided by the second cutting process is rectangular, it is preferable that an area of a first side face 10SS1 fixed to the second substrate (the dicing tape 80A) is larger than an area of a second side face 10SS2 orthogonal to the first side face 10SS1.

For example, in each image pickup unit 1, since the area S1 of the side faces 10SS1 and 10SS3 is 1.05 mm², and the area S2 of the side faces 10SS2 and 10SS4 is 0.75 mm², it is preferable that the side face 10SS1 or 10SS3, which is the first side face, is fixed to the dicing tape 80A.

### <Step S18>

The cut image pickup units 1 are removed from the dicing tape 80A which is the second substrate.

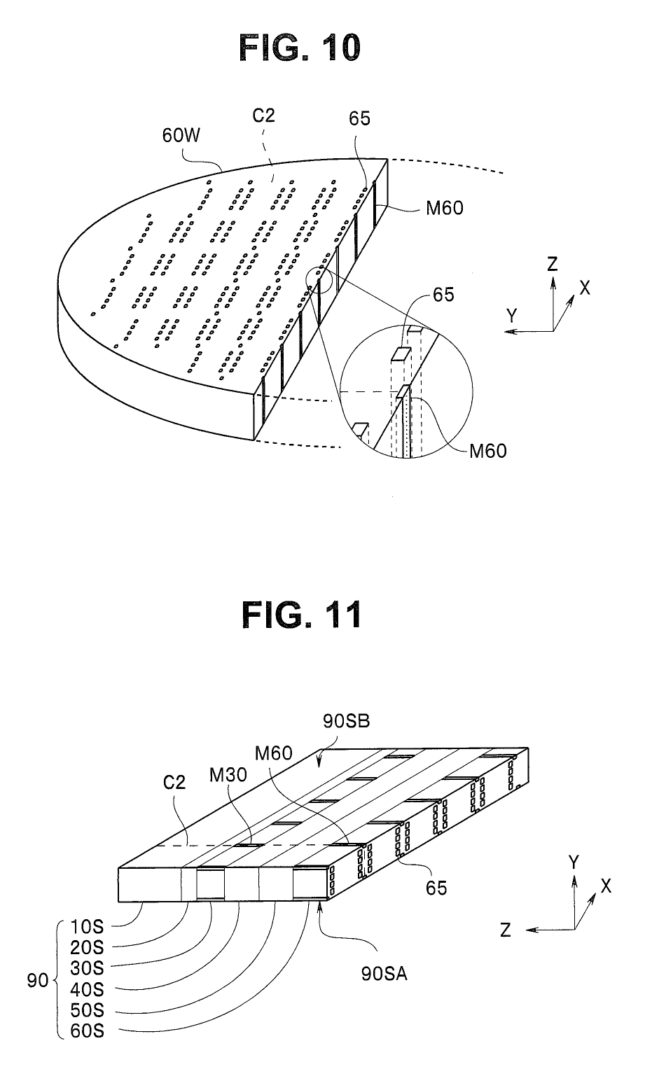

Note that it is preferable that, on any of the device wafers, for example, on the device wafer 60W, alignment marks M60 in a configuration almost the same as a configuration of the through wirings 65 are arranged as shown in FIG. 10. The alignment marks M60 indicate positions of the second cutting lines C2.

The alignment marks M60 are arranged simultaneously with the through wirings 65, and recess portions (through holes) of the device wafer 60W are filled with the same material as material of the through wirings 65, for example, copper. Note that the alignment marks M60 and the through wirings 65 may be different in cross-sectional area and cross-sectional shape.

As shown in FIG. 11, on a cutting surface of the slice body 90 (60S), cutting surfaces of the alignment marks M60 indicating the positions of the second cutting lines C2 are exposed by the first cutting process. The alignment marks M60 or the like in almost the same configuration as a configuration of the through wirings 65 can be fabricated simultaneously with the through wirings 65, which is especially preferable.

In the slice body 90 shown in FIG. 11, since alignment marks M30 are also arranged on the device wafer 30W, the alignment marks M30 are also exposed on the cutting surface of the slice body 90.

When the alignment marks indicating the positions of the second cutting line C2 are exposed on the cutting surface of the slice body 90, the cutting process is easier. Note that it is sufficient if the alignment marks are arranged on at least one device wafer.

Note that when alignment marks are arranged on each of the plurality of device wafers, the plurality of alignment marks may indicate different positions of the second cutting lines C2 because of lamination errors of the device wafers. In this case, for example, the second cutting process is performed based on average positions or the like of the positions of the second cutting lines C2 indicated by the plurality of alignment marks.

Note that alignment marks are required only to be exposed on the cutting surface of each slice body 90 by the first cutting process and are not required to be penetrated through a device wafer. The alignment marks may be arranged on a surface of a device wafer. A width of the alignment marks may be wider than a width of a cutting margin by the first cutting process, and cutting surfaces of the alignment marks may be exposed on side faces of two slice bodies 90 to be adjoined by cutting.

### Modifications of First Embodiment

Next, image pickup units 1A to 1C of modifications of the first embodiment will be described. Since the image pickup units 1A to 1C are similar to the image pickup unit 1 and have the same effects, the same reference numerals will be given to the same components, and description of the components will be omitted.

### First Modification of First Embodiment

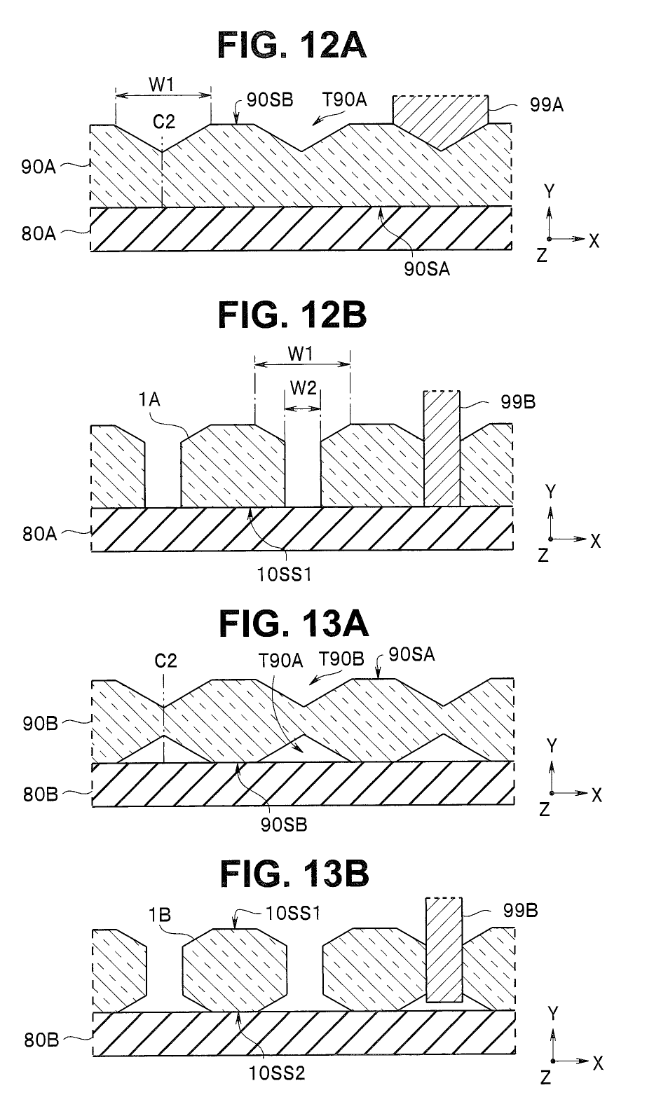

As shown in FIG. 12A, in the case of the image pickup unit 1A of the first modification, a first groove formation process of forming first grooves T90A with a V-shaped cross section along the second cutting lines C2 is further provided before the second cutting process.

That is, on each slice body 90A the cutting surface 90SA of which is fixed to the second substrate (the dicing tape 80A), the first grooves T90A with an opening width of W1 are formed using a dicing blade 99A with a V-shaped cross section.

Next, as shown in FIG. 12B, the slice body 90A is cut into image pickup units 1A using such a dicing blade 99B that a cutting margin, a space to be lost by cutting, is W2, that is, a dicing blade 99B with a width of W2. That is, the width W1 of an upper part is wider than the width W2 of a lower part in the cutting margin.

Since side faces of each image pickup unit 1A are chamfered, and a cross section is hexagonal, a volume is smaller than a volume of the image pickup unit 1, and arrangement into a small space of the rigid distal end portion 9A is easy. Other members can be accommodated in a space of the cutting margin. Therefore, a diameter of the image pickup unit 1A is small.

### Second Modification of First Embodiment

In a method for manufacturing the image pickup units 1B of a second modification, first, the first grooves T90A are formed on the cutting surface 90SB of each slice body 90A the cutting surface 90SA of which is fixed to the second substrate (the dicing tape 80A) using the dicing blade 99A with a V-shaped cross section as in the case of the image pickup units 1A (same as FIG. 12A).

Then, the slice body 90A is removed from the second substrate (the dicing tape 80A), and the cutting surface 90SB opposite to the cutting surface 90SA fixed to the second substrate is adhesively fixed to a third substrate (a dicing tape 80B). Then, as shown in FIG. 13A, a slice body 90B is fabricated, in which second grooves T90B are formed on the cutting surface 90SA of the slice body 90A fixed to the third substrate (the dicing tape 80B) using a dicing blade 99B with a V-shaped cross section.

Next, as shown in FIG. 13B, the slice body 90B is cut into image pickup units 1B using the dicing blade 99B.

All side faces of each image pickup unit 1B are chamfered, and cross sections are octagonal. Therefore, a volume is further smaller than the volume of the image pickup unit 1A, and arrangement into the small space of the rigid distal end portion 9A is easier.

### Third Modification of First Embodiment

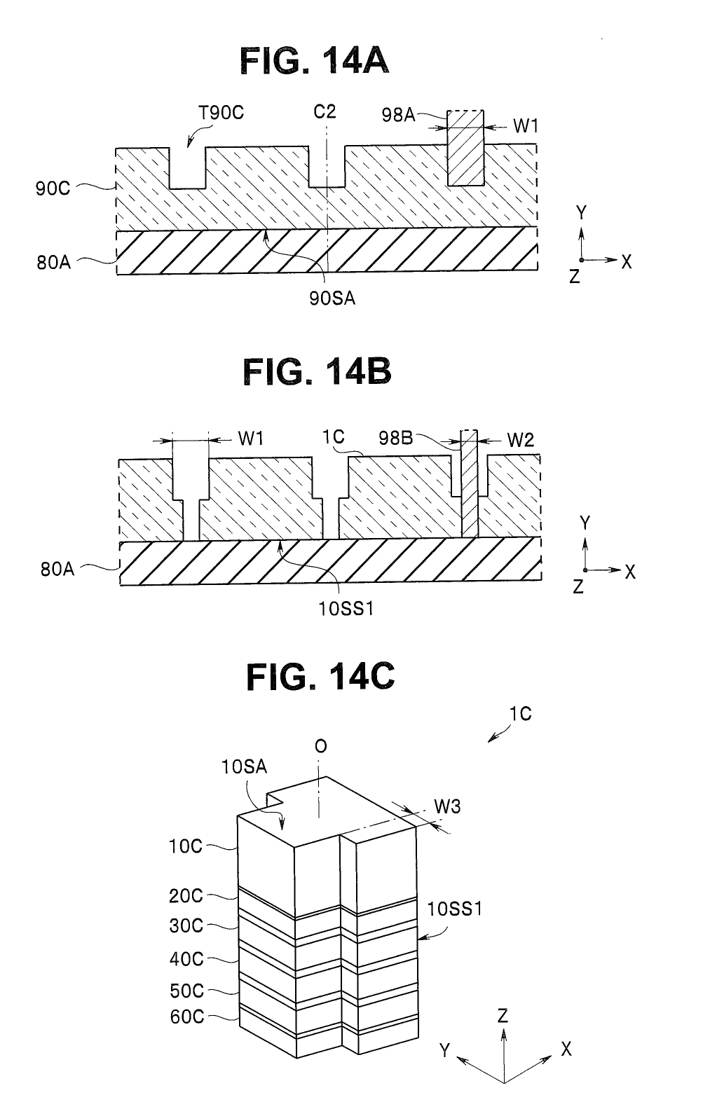

As shown in FIG. 14A, in a method of manufacturing the image pickup units 1C of a third modification, first grooves T90C are formed on each slice body 90C fixed to the second substrate (the dicing tape 80A) using a dicing blade 98A with the width of W1.

Next, as shown in FIG. 14B, the slice body 90C is cut into image pickup units 1C using a dicing blade 98B with the width of W2 narrower than W1. Therefore, the width W1 of an upper part is wider than the width W2 of a lower part in a cutting margin.

As shown in FIG. 14C, since the second cutting process for the image pickup units 1C is step dicing performed using the two kinds of blades 98A and 98B with different widths, parallel projecting parts are formed on side faces of each image pickup unit 1C. A height W3 of the projecting parts is (W1-W2).

It is easy to accurately arrange the image pickup unit 1C in a long axis direction of another member, for example, the rigid distal end portion 9A with the projecting parts used as a guide.

### Second Embodiment

Since an optical unit for endoscope of a second embodiment is similar to the image pickup units 1 to 1C and has the same effects, the same reference numerals will be given to the same components, and description of the components will be omitted.

The optical unit for endoscope of the second embodiment is a lens unit 2D in which a plurality of optical elements 10D to 50D are laminated.

The lens unit 2D is fabricated by cutting a bonded wafer in which a plurality of device wafers each of which includes a plurality of devices are laminated as in the case of the image pickup unit 1 and the like.

That is, a method for manufacturing the lens units 2D includes: a process of fabricating a plurality of lens device wafers (optical element wafers), each of the plurality of lens device wafers including a plurality of lens devices; a process of laminating the plurality of lens device wafers to fabricate a bonded wafer; a first fixation process of fixing a main face of the bonded wafer to a first substrate; a first cutting process of cutting the bonded wafer along mutually parallel first cutting lines to divide the bonded wafer into slice bodies; a process of removing the plurality of slice bodies from the first substrate; a second fixation process of fixing cutting surfaces of the slice bodies to a second substrate; and a second cutting process of cutting the slice bodies along mutually parallel second cutting lines orthogonal to the first cutting lines to divide the slice bodies into the lens units 2D with a light incident surface area of 1 mm² or smaller.

The area S2 of the side face 10SS1 of each lens unit 2D fixed to the second substrate 80A is larger than the area S1 of the light incident surface 10SA.

Since the area of fixation of each of the lens units 2D to the second substrate 80A is large, it does not happen during cutting that the lens units 2D which have been cut come off from the dicing tape 80A and are scattered or that it is not possible to cut along a desired cutting line, and, therefore, productivity of the lens units 2D is high.

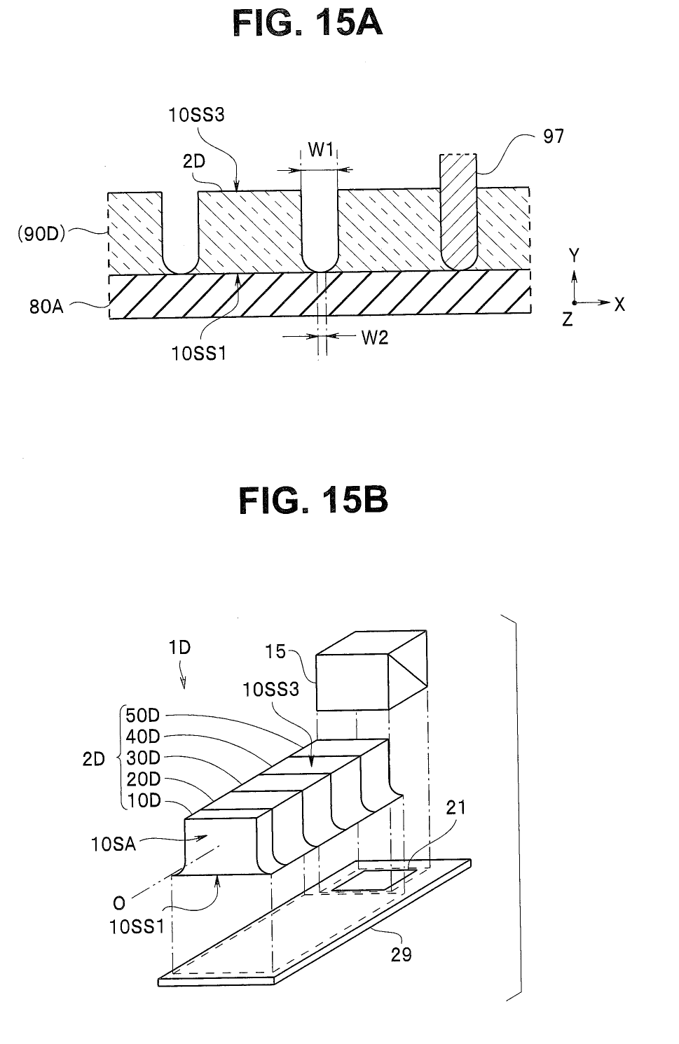

Furthermore, as shown in FIG. 15A, each slice body 90D fixed to the second substrate (the dicing tape 80A) is divided into the lens units 2D using a dicing blade 97 with a U-shaped cross section.

The width W1 of an upper part is wider than the width W2 of a lower part in the cutting margin in the second cutting process. In other words, the area S1 of the side face 10SS1 of the lens unit 2D is larger than the area S3 of the side face 10SS3.

As shown in FIG. 15B, the side face 10SS1 of the lens unit 2D is attached to an image pickup substrate 29 on which the light receiving portion 21 is formed and constitutes an image pickup unit 1D. Light incident from the light incident surface 10SA is incident on the light receiving portion 21 via a prism 15.

Productivity of the lens unit 2D is higher because an area of adhesion to the image pickup substrate 29 is larger. In the image pickup unit 1D in which the lens unit 2D is adhesively fixed to the image pickup substrate 29, it is possible to secure the area of adhesion to the image pickup substrate 29, secure a space in an upward part of the image pickup substrate 29 (a side face part of the lens unit 2D), and enable downsizing of the image pickup unit 1D and downsizing of an endoscope.

### Modification of Second Embodiment

Since lens units 2E and 2F of first and second modifications of the second embodiment are similar to the lens unit 2D and have the same effects, the same reference numerals will be given to the same components, and description of the components will be omitted.

### First Modification of Second Embodiment

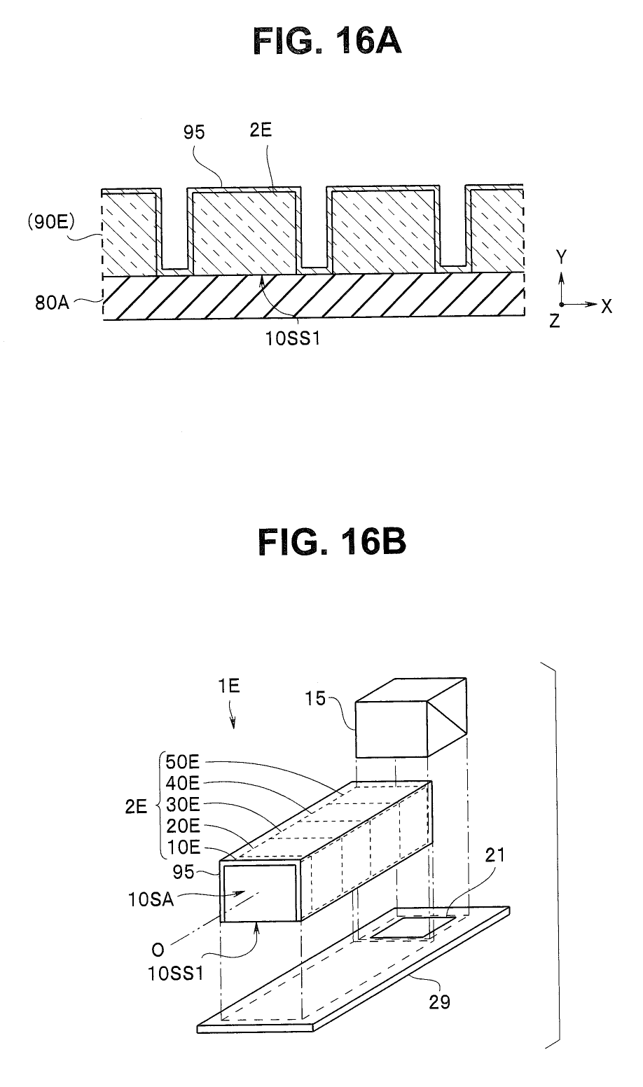

As shown in FIG. 16A, in a method for manufacturing the lens units 2E of the first modification, a process of coating a light shielding film 95 on an exposed surface of each of cut slice bodies 90E (the lens units 2E), which is not fixed to the dicing tape 80A which is a second substrate, is further provided after the second cutting process.

The light shielding film 95 with a thickness of 10 μm which is, for example, made of metal such as Cr or Ni is coated by a sputtering method or an evaporation method. The light shielding film 95 prevents external light from entering an optical path of the lens unit 2E.

As shown in FIG. 16B, three side faces among four side faces of the lens unit 2E of the first modification are covered with the light shielding film 95. Since the side face 10SS1 which is not covered with the light shielding film 95 is attached to the image pickup substrate 29, it does not happen that external light enters.

Material, a thickness and a coating method of the light shielding film 95 are appropriately selected. Note that instead of the light shielding film 95, an inorganic insulating film of silicon oxide, silicon nitride or the like having a function of a barrier layer against water may be coated on the side faces. Furthermore, the light shielding film 95 and the inorganic insulating film may be coated.

### Second Modification of Second Embodiment

Four side faces of each lens unit 2F of the second modification are covered with the light shielding films 95 (95A and 95B).

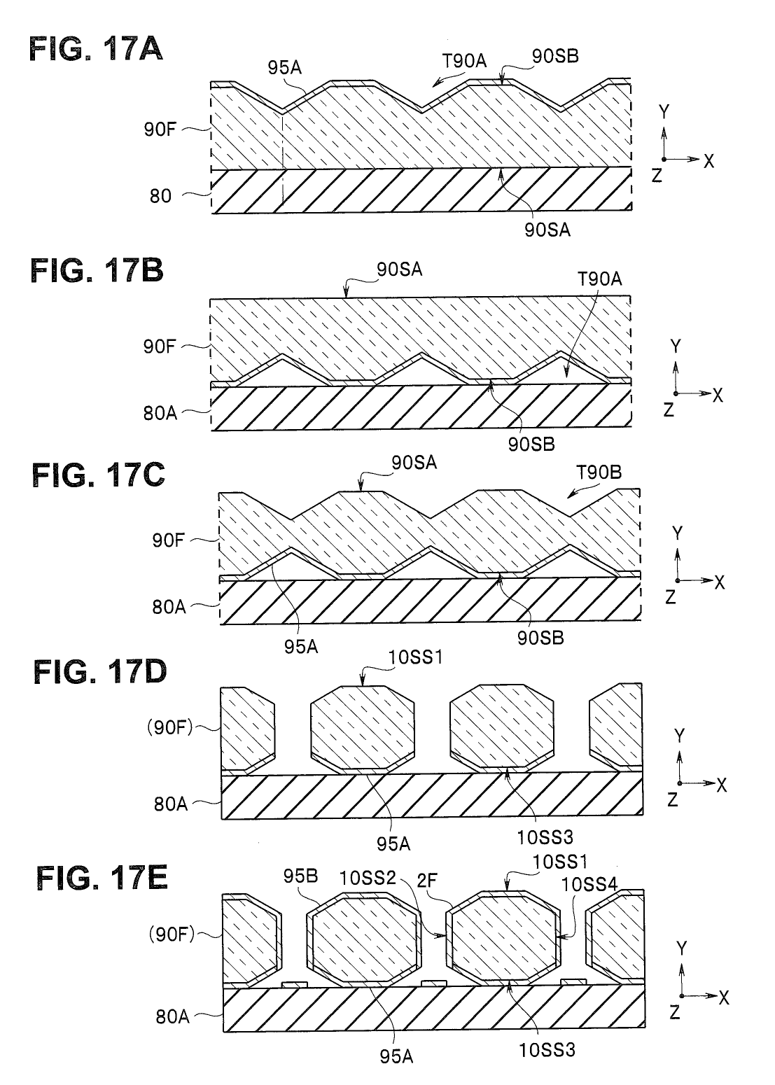

As shown in FIG. 17A, the first V grooves T90A are formed on the cutting surface 90SB of each slice body 90F fixed to the first substrate (the dicing tape 80), and the light shielding film 95A is coated.

As shown in FIG. 17B, the slice body 90F is removed from the dicing tape 80 which is the first substrate, and the cutting surface 90SB is fixed to the dicing tape 80A which is the second substrate.

As shown in FIG. 17C, the same second V grooves T90B as the first V grooves T90A are formed on the cutting surface 90SA of the slice body 90F fixed to the second substrate (the dicing tape 80A).

Then, as shown in FIG. 17D, the slice body 90F is divided by cutting.

As shown in FIG. 17E, the light shielding film 95B is coated on an exposed surface of the cut slice body 90F, which is not fixed to the dicing tape 80A, as in the case of the light shielding film 95A. Note that materials and thicknesses of the light shielding film 95A and the light shielding film 95B may be the same or may be different.

Note that in the image pickup units 1, and 1A to 1C, light shielding films or the like can be coated on the side faces by a method similar to the method for manufacturing the lens units 2E or 2F. Further, in the lens units 2D to 2F, alignment marks can be formed by a method similar to the methods for manufacturing the image pickup units 1A to 1C. The alignment marks can be formed by vapor deposited films having an aperture function, which is formed on the lens device wafers of the lens units 2D to 2F. The alignment marks may be formed by a resin mold for forming a lens device.

It goes without saying that an endoscope provided with any of the image pickup units 1A to 1C or any of the lens units 2D to 2F in a rigid distal end portion of an insertion portion has the same effects as the endoscope 9 and has effects of the respective units.

The present invention is not limited to the embodiments described above, and various modifications, alterations and the like can be made within a range not departing from the spirit of the present invention.

What is claimed is:

1. A method for manufacturing optical units for endoscope, comprising:
a step of fabricating a plurality of device wafers including at least one optical element wafer, each of the plurality of device wafers including a plurality of devices;
a step of laminating the plurality of device wafers to fabricate a bonded wafer;
a first fixation step of fixing a main face of the bonded wafer to a first substrate;
a first cutting step of cutting the bonded wafer along mutually parallel first cutting lines to divide the bonded wafer into slice bodies;
a step of removing the slice bodies from the first substrate;
a second fixation step of fixing cutting surfaces of the slice bodies to a second substrate;
a second cutting step of cutting the slice bodies along mutually parallel second cutting lines orthogonal to the first cutting lines to divide the slice bodies into the optical units for endoscope; and
a step of removing the optical units for endoscope from the second substrate; wherein
an area of a side face of each of the optical units for endoscope is larger than an area of a light incident surface, the side face being fixed to the second substrate and being orthogonal to the light incident surface.

2. The method for manufacturing the optical units for endoscope according to claim 1, wherein the area of the side face of each of the optical units for endoscope divided by the second cutting step is larger than an area of a second side face orthogonal to the side face, the side face being fixed to the second substrate.

3. The method for manufacturing the optical units for endoscope according to claim 1, wherein alignment marks indicating positions of the second cutting lines are exposed on the cutting surfaces of the slice bodies by the first cutting step.

4. The method for manufacturing the optical units for endoscope according to claim 1, further comprising a step of coating exposed surfaces not fixed to the second substrate with at least either an inorganic insulating film or a light shielding film after the second cutting step.

5. The method for manufacturing the optical units for endoscope according to claim 1, wherein each of the optical units for endoscope is a rectangular parallelepiped.

6. The method for manufacturing the optical units for endoscope according to claim 1, wherein a width of an upper part is wider than a width of a lower part in a cutting margin in the second cutting step.

7. The method for manufacturing the optical units for endoscope according to claim 6, wherein
the second cutting step is step dicing performed using two kinds of blades with different widths; and
parallel projecting parts are formed on side faces.

8. The method for manufacturing the optical units for endoscope according to claim 6, further comprising a first groove formation step of forming first grooves with a V-shaped cross section along the second cutting lines before the second cutting step.

9. The method for manufacturing the optical units for endoscope according to claim 1, wherein
the optical element is a cover glass element, and each of the optical units for endoscope is an image pickup unit in which the cover glass element, an image pickup device and a plurality of semiconductor devices are laminated.

10. The method for manufacturing the optical units for endoscope according to claim 1, wherein each of the optical units for endoscope is a lens unit in which a plurality of optical elements are laminated.

11. An endoscope comprising an optical unit for endoscope in a rigid distal end portion of an insertion portion, wherein
the optical unit for endoscope is manufactured by a manufacturing method comprising:
a step of fabricating a plurality of device wafers including at least one optical elements wafer, each of the plurality of device wafers including a plurality of devices;
a step of laminating the plurality of device wafers to fabricate a bonded wafer;
a first fixation step of fixing a main face of the bonded wafer to a first substrate;
a first cutting step of cutting the bonded wafer along mutually parallel first cutting lines to divide the bonded wafer into slice bodies;
a step of removing the slice bodies from the first substrate;
a second fixation step of fixing cutting surfaces of the slice bodies to a second substrate;
a second cutting step of cutting the slice bodies along mutually parallel second cutting lines orthogonal to the first cutting lines to divide the slice bodies into the optical units for endoscope; and
a step of removing the optical units for endoscope from the second substrate; and
an area of a side face of each of the optical units for endoscope is larger than an area of a light incident surface, the side face being fixed to the second substrate and being orthogonal to the light incident surface.
